(19) United States
(12) Patent Application Publication
(10) Pub. No.: US 2009/0018648 A1
(43) Pub. Date: Jan. 15, 2009

(54) STENT WITH A COATING

(75) Inventor: Eric Wittchow, Nuernberg (DE)

Correspondence Address: POWELL GOLDSTEIN LLP, ONE ATLANTIC CENTER FOURTEENTH FLOOR, 1201 WEST PEACHTREE STREET NW, ATLANTA, GA, 30309-3488, US

(73) Assignee: BIOTRONIK VI PATENT AG, Baar (CH)

(21) Appl. No.: 12/171,511

(22) Filed: Jul. 11, 2008

(30) Foreign Application Priority Data
Jul. 13, 2007 (DE) 10 2007 032 686.8

(51) Int. Cl. A61F 2/82

(52) U.S. Cl. 623/1.46

(57) ABSTRACT

A stent of a metallic base body with a selenium-containing coating or filling of a cavity.

### PRIORITY CLAIM AND CROSS REFERENCE TO RELATED APPLICATION

This patent application claims priority to German Patent Application No. 10 2007 032 686.8, filed Jul. 13, 2007, the disclosure of which is incorporated herein by reference in its entirety. This application is related to co-pending U.S. patent application Ser. No. ______, Attorney Docket No. 149459.00035, filed Jul. 11, 2008, and entitled Stent With A Coating Or Filling Of A Cavity, the disclosure of which is incorporated herein by reference in its entirety.

### FIELD

The present disclosure relates to a stent having a metallic base body and a coating.

### BACKGROUND

Implantation of stents has proven to be one of the most effective therapeutic measures in treatment of vascular diseases. The purpose of stents is to assume a supporting function in hollow organs of a patient. Stents of a traditional design therefore have a filigree supporting structure of metallic struts, which are initially in a compressed form for introducing them into the body and then are widened at the site of application. One of the main areas of application of such stents is for permanent or temporary widening of vascular occlusions and keeping them open, in particular obstructions (stenoses) of the myocardial vessels. In addition, there are also aneurysm stents which serve to support damaged vascular walls, for example.

Stents have a circumferential wall of a sufficient supporting force to keep the constricted vessel open to the desired extent and have a tubular base body through which the blood can flow unhindered. The supporting circumferential wall is usually formed by a mesh-like supporting structure which allows the stent to be inserted in a compressed state with a small outside diameter up to the constriction in the respective blood vessel that is to be treated and widened there, for example, with the help of a balloon catheter, so that the blood vessel has the desired enlarged inside diameter.

The stent has a base body of an implant material. An implant material is a nonviable material that is used for applications in medicine and interacts with biological systems. The basic prerequisite for use of a material as an implant material, which in the intended purpose is in contact with the bioenviromnent, is its biocompatibility. Biocompatibility is understood to be the ability of a material to induce an appropriate tissue reaction in a specific application. This includes adaptation of the chemical, physical, biological and morphological surface properties of an implant to the recipient tissue with the goal of a clinically desired interaction. The biocompatibility of the implant material also depends on the chronological course of the reaction of the biosystem into which it is implanted. Thus irritation and inflammation may occur for a relatively short time and may lead to tissue changes. Biological systems thus react differently, depending on the properties of the implant material. According to the reaction of the biosystem, the implant materials may be subdivided into bioactive, bioinert and degradable/absorbable materials.

For the purposes of the present disclosure, only metallic implant materials are of interest for stents. Biocompatible metals and metal alloys for permanent implants include stainless steels (e.g., 316L), cobalt master alloys (e.g., CoCr (L605), CoCrMo casting alloys, CoCrMo forge alloys, CoCrWNi forge alloys and CoCrNiMo forge alloys), pure titanium and titanium alloys (e.g., cp titanium, TiAl6V4 or TiAl6Nb7) and gold alloys. In the area of biocorrodible stents, the use of magnesium or pure iron as well as biocorrodible master alloys of the elements magnesium, iron, zinc molybdenum and tungsten is proposed.

A biological reaction to metallic elements depends on the concentration, exposure time and type of administration. The presence of an implant material often leads to inflammation reactions, in which the triggering factors may be mechanical irritation, chemical substances as well as metabolites. The inflammation process is usually accompanied by the migration of neutrophilic granulocytes and monocytes through the vessels, migration of lymphocyte effector cells, forming specific antibodies to the inflammation stimulus, activation of the complement system with the release of complement factors that act as mediators and ultimately the activation of blood coagulation. An immunological reaction is usually closely associated with the inflammation reaction and may lead to sensitization and development of an allergy. Known metal allergens include, for example, nickel, chromium and cobalt, which are also used as alloy components in many surgical implants. An important problem in stent implantation in blood vessels is in-stent restenosis due to overshooting neointimal growth, which is caused by a great proliferation of arterial smooth muscle cells and a chronic inflammation reaction.

It is known that a higher measure of biocompatibility and thus an improvement in restenosis rate can be achieved if metallic implant metals are provided with coatings of materials that are especially biocompatible. These materials are usually of an organic or a synthetic polymer type and to some extent may also be of natural origin. Additional strategies to prevent restenosis are concentrated on inhibiting proliferation through medication, e.g., treatment with cytostatics.

Despite the progress that has been achieved, there is still a great need for achieving a better integration of the stent into its biological environment and thereby lowering the restenosis rate.

### SUMMARY

The present disclosure describes several exemplary embodiments of the present invention.

The present disclosure provides a stent, comprising a) a metallic base body; and b) either a selenium-containing coating or a selenium-containing filling of a cavity.

According to a first aspect of the present disclosure, one or more of the problems described above are solved by a stent comprised of a metallic base body and a selenium-containing coating or filling of a cavity.

The present disclosure is based on the finding that, in a healthy body, there is an equilibrium between cell reproduction (cell proliferation) and cell death (apoptosis). If a restenosis occurs after implantation of a stent, the equilibrium between the two processes is disturbed and proliferation gains the upper hand over natural cell death. Previous strategies for preventing restenosis have been based on inhibition of proliferation. However, porcine histological preparations of stenosed vessels have not shown elevated levels of proliferation markers in comparison with the surrounding tissue. Bauriedel (J. Vasc. Res. 2004; 41(6); 525-534) performed immunohistological examinations on atherectomized specimens from patients with symptomatic in-stent restenosis and found that programmed cell death (apoptosis) is significantly reduced in older lesions in comparison with primary atheromas. This supports the assumption that apoptosis occurs less effectively than in healthy tissue. This is where the present invention begins. The imbalance between cell proliferation and apoptosis is to be balanced by increasing the rate of apoptosis. The advantage in comparison with inhibited proliferation, which equally affects unwanted neointimal cells and essential endothelial cells, is unhindered cell proliferation around the stent. If endothelial cell proliferation is disturbed, there is a delay in tissue coverage of the stent, increased thrombosis and a risk of fatal vascular occlusion.

It has been found that the use of elemental selenium or selenium compounds as components of a coating or filling of a cavity of a metallic stent leads to an increased apoptosis. The positive influence of selenium on the mechanism of action on which apoptosis is based is still largely unexplained. Presumably the caspase-3 enzyme which triggers the apoptotic process directly is activated.

Preferably inorganic selenium compounds are used, in particular selenium dioxide (SeO2), selenium disulfide (SeS2), selenides (especially preferably MgSe), selenites, selenates or selenophosphates (H3SePO4). Inorganic selenium compounds usually have a greater thermal stability in comparison with organic selenium compounds, so the production and sterilization of this stent are simplified. Nevertheless, organic selenium compounds such as selenocysteine, selenodiglutathione, selenomethothionine and other selenoproteins may also be used.

The apoptosis-stimulating material may be part of a coating, or the coating may consist entirely of the material. In the former case, a selenium salt in pulverized form, for example, may be embedded in a biodegradable polymer matrix. Furthermore, it may be part of the electrolyte in production of a magnesium conversion layer (MAGOXID, MAGPASS; BIOXID) on a stent made of a biocorrodible magnesium alloy, so that it is embedded in the conversion layer and is released by degradation thereof. As a rule, the coating is applied directly to the base body of the stent. However, intermediate layers may also be present, if necessary. Alternatively, the apoptosis-stimulating material may be part of a cavity filling. The cavity is usually at the surface of the stent. In the case of stents with a biodegradable base body, the cavity may also be arranged in the interior of the base body so that the material is released only after being exposed. The coating or filling preferably comprises 0.1 to 20 μg free or bound selenium per 1 mm stent length.

The coating or filling preferably additionally comprises arsenic or a compound containing arsenic. It has been found that a combination of selenium and arsenic lowers the toxicity so that unwanted side reactions are reduced. Selenium thus has a positive effect on the arsenic-induced cytotoxicity and an influence on cell viability. The biocompatibility of arsenic can therefore be improved, another element promoting apoptosis.

According to another exemplary embodiment the metallic base body has a porous surface which is covered with the selenium-containing coating. In other words, the accessible pores of the porous surface of the metallic base body are covered/filled with the selenium-containing coating. In this way, the contact area with the biological system can be increased and the effects on apoptosis can be potentiated as desired according to the present disclosure.

The metallic basic structure is preferably made of magnesium, a biocorrodible magnesium alloy, pure iron, a biocorrodible iron alloy, a biocorrodible tungsten alloy, a biocorrodible zinc alloy or a biocorrodible molybdenum alloy. The aforementioned biocorrodible metallic materials are usually mostly inert chemically with respect to selenium and selenium compounds so that no negative effect on the degradation of the stent need be expected.

A combination in which the metallic basic structure of the stent comprises a biodegradable magnesium alloy and the coating comprises MgSe or comprises MgSe is especially preferred.

Alloys and elements are referred to as biocorrodible (or biodegradable) in the sense of this disclosure when a degradation/conversion takes place in a physiological environment so that the part of the implant comprised of the material is entirely or at least predominately no longer present.

For purposes of the present disclosure, the terms magnesium alloy, iron alloy, zinc alloy, molybdenum alloy or tungsten alloy refer primarily to a metallic structure whose main component is magnesium, iron, zinc, molybdenum or tungsten. The main component is the alloy component present in the greatest amount by weight in the alloy. The amount of the main component is preferably more than 50 wt %, in particular, more than 70 wt %. The composition of the alloy is to be selected so that it is biocorrodible. Synthetic plasma as defined in EN ISO 10993-15:2000 (composition NaCl 6.8 g/L, CaCl2 0.2 g/L, KCl 0.4 g/L, MgSO₄ 0.1 g/L, NaHCO₃ 2.2 g/L, Na₂HPO₄ 0.126 g/L, NaH₂PO₄ 0.026 g/L) for biocorrosion studies is used as the test medium for testing the corrosion behavior of an alloy. A sample of alloy to be tested is therefore stored in a sealed sample container with a defined amount of test medium at 37° C. The samples are removed at intervals (based on the corrosion behavior to be expected) of a few hours to several months and then tested for traces of corrosion by known methods. The artificial plasma according to EN ISO 10993-15:2000 comprises a medium resembling blood and thus permits reproducible simulation of a physiological environment in the sense of the present disclosure.

### DETAILED DESCRIPTION

The present disclosure will be explained in greater detail below on the basis of exemplary embodiments.

### EXAMPLES

A stent of the biodegradable magnesium alloy WE43 (according to ASTM) is degreased and dried. The stent may have cavities at its surface. The coating is performed as follows:

### Example 1

### Coating the Stent with Polymer Matrix Containing Active Ingredient

A 0.05 to 0.4% solution of a poly(orthoester) is prepared in dry THF, which is in turn prepared from 3,9-diethylidene-2,4,8,10-tetraoxaspiro[5.5]-undecane and trans-cyclohexanedimethanol, 1,6-hexanediol, triethylene glycol and triethylene glycol glycolide (molar ratio: 15/40/40/5). The stent is cleaned to remove dust and residues and clamped in a suitable stent coating apparatus. A clear 10% solution of selenomethionine in THF is added to the polymer solution in such a way that the polymer and the active ingredient are in a weight ratio range of 30/70 to 80/20 (preferably 60/40). Using an airbrush system, the rotating stent is half coated under constant ambient conditions (room temperature, 42% atmospheric humidity). At a nozzle distance of 20 mm, an 18-mm-long stent is coated after approximately 10 minutes. The coating composition is to be selected so that the stent comprises 0.1 μg-10 μg (preferably 1 μg) selenomethionine/mm. After reaching the intended coating weight, the stent is dried for 5 minutes at room temperature before the uncoated side is coated in the same way after rotating the stent and clamping it again. The completely coated stent is dried for 24 hours at 80° C. in a vacuum oven.

### Example 2

### Coating a Stent Provided with Cavities with Elemental Selenium

A saturated ethanolic solution of powdered selenious acid (H₂SeO₃) is prepared and the stent provided with the cavities is suspended in the solution at room temperature on a suitable device for 3-5 min in such a way that the stent is wetted on all sides. The suspension for hanging the stent expediently comprises a magnesium wire because other more noble metals would form a local element with magnesium and plastic often cannot withstand the temperatures for the subsequent sintering step. The stent which has a reddish color at the surface is removed from the solution and cautiously blown off with compressed air. The stent is suspended on the same magnesium wire for 2 minutes in a 230° C. annealing furnace under an air atmosphere, whereupon the reddish selenium melts, partially penetrates into the cavities and forms a thin metallic film after solidifying at room temperature. The amount of biologically active substance can be determined gravimetrically. The release of selenium can be modified by applying a polymer top layer.

### Example 3

### Modification of a Stent with a Conversion Layer Containing Selenium

The stent is cleaned for 1 minute in a saturated solution of KOH in isopropanol and rinsed briefly with a generous amount of deionized water. Then anodic oxidation is performed in an aqueous electrolyte bath containing 30 g/L H₂SeO₃ (selenious acid), 55 g/L H₃PO₄ (phosphoric acid) and 300 g/L hexamethylenetetramine. The pH is adjusted to 8.5 with NH₄OH. Anodic oxidation is performed for 5 minutes at 20° C. using a pulsed direct current with a current density of 1.1 A/dm² and a voltage increasing to 240 V. The thickness of the resulting selenium-containing layer is 5 μm.

All patents, patent applications and publications referred to herein are incorporated by reference in their entirety.

What is claimed is:

1. A stent, comprising:
a) a metallic base body; and
b) either a selenium-containing coating or a selenium-containing filling of a cavity.

2. The stent of claim 1, wherein the coating or filling comprises elemental selenium, SeO2, SeS2, selenides, selenites, selenates and selenophosphates.

3. The stent of claim 1, wherein the metallic basic structure of the stent comprises a material selected from the group consisting of magnesium, a biocorrodible magnesium alloy, pure iron, a biocorrodible iron alloy, a biocorrodible tungsten alloy, a biocorrodible zinc alloy and a biocorrodible molybdenum alloy.

4. The stent of claim 3, wherein the metallic basic structure of the stent is made of a biodegradable magnesium alloy.

5. The stent of claim 1, wherein the coating or filling comprises either 0.1 to 20 μg free or bound selenium per 1 mm of stent length.

6. The stent of claim 1, wherein the coating or filling additionally comprises either arsenic or a compound that contains arsenic.

7. The stent of claim 3, wherein the coating either comprises MgSe or is made of MgSe.
